(19) United States
(12) Patent Application Publication
(10) Pub. No.: US 2009/0240133 A1
(43) Pub. Date: Sep. 24, 2009

(54) Apparatus and method for non-invasive, in-vivo, thoracic radio interrogation

(75) Inventors: Robert Friedman, Las Vegas, NV (US); Andrew Pal, Las Vegas, NV (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: NonInvasive Medical Technologies, Inc., Las Vegas, NV (US)

(21) Appl. No.: 12/383,357

(22) Filed: Mar. 23, 2009

Related U.S. Application Data
(63) Continuation-in-part of application No. PCT/US2007/020473, filed on Sep. 21, 2007.
(60) Provisional application No. 60/846,403, filed on Sep. 21, 2006.
(60) Provisional application No. 60/846,402, filed on Sep. 21, 2006.
(60) Provisional application No. 60/973,985, filed on Sep. 20, 2007.

(51) Int. Cl. A61B 5/05

(52) U.S. Cl. 600/407

(57) ABSTRACT

A radio apparatus and method for non-invasive, thoracic radio interrogation of a subject for the collection of hemodynamic, respiratory and/or other cardiopulmonary related data from the subject includes a antenna positionable proximally to the subject, a radio transmitter transmitting an unmodulated radio interrogation signal of a predetermined fixed frequency at a safe level of about 1 milliwatt or less from the antenna into the subject and a radio receiver capturing through the antenna, reflections of the transmitted radio interrogation signal returned from the subject. A Doppler component of the reflections contains the data that can be extracted from the captured reflections.

### CROSS REFERENCE TO RELATED APPLICATIONS

The present application is a continuation-in-part of PCT/US2007/020473 filed Sep. 21, 2007, entitled “Apparatus and Method for Non-invasive Thoracic Radio Interrogation” and claims priority from U.S. Patent application Nos. 60/846,403 entitled “Method and Apparatus for Non-Invasive Bio Impedance Determination”, filed Sep. 21, 2006, U.S. Provisional application No. 60/846,402 entitled “Method for Conditioning Radio Signal Returns from Thoracic Components for Extractions of Cardiopulmonary Data”, filed Sep. 21, 2006, and U.S. Provisional application No. 60/973,985, entitled “Apparatus and Method for Non-Invasive Thoracic Radio Interrogation”, filed Sep. 20, 2007. All are incorporated by reference herein in their entireties.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms to the extent and under the provisions as provided for by Contract No. DAH001-05-S-0144 awarded by the U.S. Air Force Special Operations Command (AFSOC).

### BACKGROUND OF THE INVENTION

Until relatively recently, hemodynamic monitoring has been limited to the critical care unit, operating room and occasionally the emergency department due to the invasive nature of the pulmonary artery catheter used, the expertise required for insertion and maintenance of the catheter, and the close vigilance required to prevent potential vital risks to the patient. Accepted invasive hemodynamic monitoring methods include the Fick method, dye indicator dilution, and thermodilution.

Contact impedance cardiography systems now available provide noninvasive monitoring of patient hemodynarnics. Unlike invasive hemodynanic monitoring with a pulmonary artery catheter, noninvasive contact impedance monitoring is sufficiently safe and easy to use that hemodynamic monitoring need no longer be restricted to care of the critically ill. Noninvasive continuous hemodynamic monitoring has utility in any clinical area, from the outpatient clinic to the critical care unit, where healthcare providers desire information regarding a patient's hemodynamic status without invasive procedure.

While contact impedance cardiography technology is a marked improvement to invasive technologies, it still has some limitations. It requires a power supply and the careful placement of several electrodes on the patient's torso. While this does not seem like a significant drawback, it has been an impediment to movement of noninvasive continuous hemodynamic monitoring from the hospital emergency room to the medical first aid arena, encompassing virtually any emergency field situation: natural disaster, or other emergency medical scenario.

### BRIEF SUMMARY OF THE INVENTION

In one aspect, the invention is an apparatus for non-invasive, in-vivo, thoracic radio interrogation of a subject to collect hemodynamic, respiratory and/or other cardiopulmonary related data from the subject that comprises an antenna sized to be positioned proximally to the subject; a radio transmitter operably connected to the antenna and configured to transmit an unmodulated radio interrogation signal of a predetermined fixed frequency through the antenna into the proximately positioned subject and a radio receiver operably connected to the antenna and configured to capture through the antenna, reflections of the transmitted radio interrogation signal returned from the subject.

In another aspect, the invention is a method for non-invasive, in-vivo, thoracic radio interrogation of a subject to collect hemodynamic, respiratory and/or other cardiopulmonary related data from the subject comprising the steps of: positioning an antenna proximally to a subject; transmitting only an unmodulated radio interrogation signal of a predetermined fixed frequency through the antenna and into the proximately positioned subject; and capturing through the antenna with an operably connected radio receiver reflections of the transmitted radio interrogation signal returned from the subject.

Other, more detailed aspects of the invention are set forth in the attached claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 depicts exemplary simulated traces of simultaneously obtained conventional, noninvasive conductive impedance signal, the time varying component of the conventional, noninvasive conductive impedance signal, a radio interrogation impedance signal of the present invention and an ECG;

FIG. 2 depicts graphically, the electrical and mechanical phases of the typical cardiac cycle as reflected in various traces;

FIG. 3 depicts diagrammatically, use of a noninvasive, in-vivo, non-contact, radio interrogation system of the present invention;

FIG. 4 is a block diagram of the presently preferred, noninvasive, radio interrogation system of FIG. 3; and

FIG. 5 is a diagram depicting the various thoracic, radio reflection surfaces in the human body.

### DETAILED DESCRIPTION OF THE INVENTION

It has been found that a radio signal can be transmitted into the body of a living subject without physical contact with the body at a low enough power level to be safe and the reflections of that signal collected with hemodynamic and other related cardio-respiratory data sufficiently detailed and accurate to at least monitor cardio-respiratory condition and changes of the subject and to quantify important cardiac functions that heretofore have had to be collected by conventional contact or invasive impedance measurement methods and equipment. More specifically, it has been found that a radio signal transmitted into a body without contact and a voltage test signal applied to the body by contact as in conventional, contact impedance systems, both undergo changes as a result of their encounters with different substances present in the human body. It further has been found that the transmitted radio signal, like the impedance voltage signal conducted through the torso in conventional contact impedance measurement, is particularly sensitive to electrically conductive substances and is modified in amplitude, phase and frequency at least in part by the dynamic changes of varying blood volume, flow velocity and possibly even alignment of the red blood cells that reflect the mechanical activity of the heart. Accordingly, like the voltage test signal used in conventional, contact impedance measurement, reflections of a transmitted radio interrogation signal carry information that permit status monitoring and even determination of at least some of the same cardiac functions.

It particular, it has been discovered that the time varying portions of the reflections of a radio interrogation signal contain cardiac function events and landmarks like those found in conventional, contact impedance voltage measurements. For that reason, an explanation of the reflected radio interrogation signal processing of the present invention can be analogized to the better-known processing of conventional contact impedance generated signals.

Impedance (Z) is the resistance to the flow of electrical current and is measured in ohms. Blood and other body fluids are excellent conductors of electricity and have low impedance, particularly compared to bone, other tissue, and air. Blood and fluid in the lungs are the most conductive substances in the thorax. Thus, larger quantities of thoracic fluid/blood lower impedance and smaller quantities result in increased impedance while larger quantities of thoracic fluid/blood provide greater radio wave reflectivity and smaller quantities result in reduced reflectivity.

Thoracic impedance (Z) measured at any instant in time is primarily dependent on blood volume, flow velocity, and even alignment of red blood cells (RBC). Increases in volume and flow during the systole phase of the cardiac cycle, reduce the measured impedance Z while decreases in blood volume and flow, and more random configuration of RBCs during the diastole phase causes an increase in impedance as blood pressure decreases after the heart has ejected blood. This time varying change in the thoracic impedance, ΔZ/Δt, represents the mechanical activity of the heart. The time average or baseline impedance, Z_{O}, predominantly reflects total thoracic fluid volume and is another important parameter in fluid management.

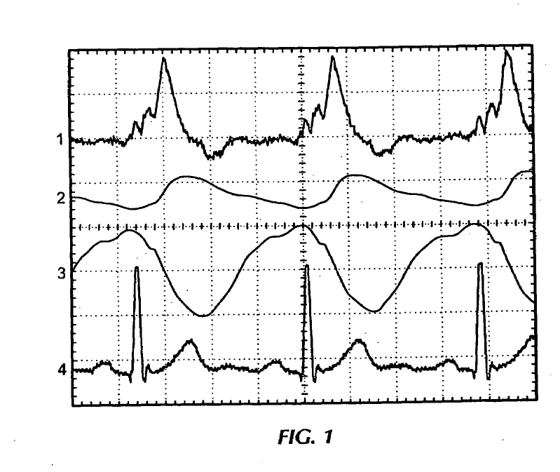

The process of the heart pumping blood is the cardiac cycle which has both mechanical and electrical phases. FIG. 1 depicts traces of various exemplary simulated signal waveforms like those that might be taken from the same subject over the same time period. The electrical phase, measured by electrocardiogram (ECG), is shown as trace 4 in FIG. 1 and reflects the electrical activity of the myocardium which triggers the mechanical activity of the heart. The ECG waveform represents the command signals through the cardiac cycle, which must have the proper timing and intensity to enable all four heart chambers heart to beat in proper sequence and pump blood effectively.

The mechanical phase of the cardiac cycle reflects the ability of the heart to efficiently pump blood. Trace 1 in FIG. 1 depicts the corresponding contact impedance cardiography waveform Zo. Trace 2 of FIG. 1 depicts the time varying change ΔZ/Δt in the Zo signal of trace 1. The contact impedance cardiography waveform data represents the mechanical phase of the cardiac cycle and contains information that is not in the ECG waveform (trace 4). Trace 3 is the radio frequency interrogation impedance (RFII) signal of the present invention as it will be subsequently defined

Conventional contact impedance cardiology measurements are normally divided into DC and AC components, representing a constant baseline cardiac impedance Z_{O}, and a time varying component ΔZ/Δt, where

Impedance (Z)=Zo+ΔZ/Δt

and the first and second order time varying components are given as,

ΔZ/Δt=dZ/dt+d²Z/dt²

Signal processing of the contact cardiac impedance waveform (trace 1 of FIG. 1) can extract vital information about the cardiovascular system's status, body hydration and even otherwise difficult to detect internal bleeding. Signal processing can combine impedance data with other common biomedical data like heart rate, blood pressure and ECG parameters for a very comprehensive real-time hemodynamic status report of the subject.

Baseline impedance Zo predominantly reflects total thoracic fluid volume. There are normal limits for men (20-30 ohms) and women (25-35 ohms) and deviation from the normal may indicate adverse conditions that actually change blood and body tissue chemistry and conductivity, such as dehydration, excess water or a lack of oxygen. Since generally the blood, tissue and bone content are constant, a slower change or trend in baseline impedance outside of the time-window for the cardiac cycle and respiration can indicate internal bleeding or other increase in extra vascular fluid predicting a life threatening situation before other symptoms may appear.

Heart rate is another major determinate of cardiac impedance. A change in heart rate is one of the first compensatory mechanisms for the heart to maintain cardiac output and oxygen transport. Moderate increases or decreases in heart rate will correspondingly increase or decrease cardiac output. If heart rate is excessively elevated diastolic filling time, preload, stroke volume and coronary artery blood flow is adversely reduced. Excessive reduction of heart rate increases diastolic filling time and may impair contractility and decrease stroke volume due to damaging over-stretching of the heart muscle fiber.

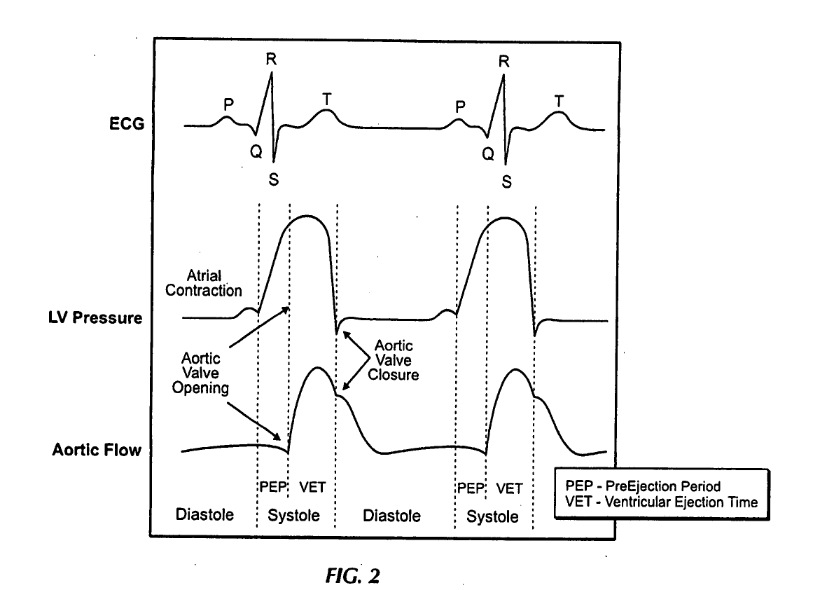

The first order change in impedance related to time, ΔZ/Δt, generates a waveform (like trace 2 in FIG. 1) that is similar to the aortic flow curve or velocity of blood flow, which is shown in trace 3 of FIG. 2 with another sample ECG (trace 1 of FIG. 2). The magnitude and rate of the impedance change is a direct reflection of left ventricular contraction further illustrated by a trace of left ventricle (“LV”) blood pressure (trace 2 of FIG. 2). When the time varying cardiac impedance waveform is combined with timing data from the cardiac cycle, such as the ECG, a signal processing timing window is created so each cardiac cycle can be evaluated in real time and separated from respiratory motion and other changes.

By including the parameters of the heart rate and blood pressure in signal processing, cardiac performance can be determined. Stroke volume (SV) is the blood volume ejected into the aorta every beat. Stroke volume is typically 60 to 120 ml/beat. Cardiac output (CO) is a function of stroke volume, the blood volume ejected with each ventricular contraction, and heart rate. Normal cardiac output at rest is considered to be 4 to 8 liters/minute (1/min). Cardiac output can be adjusted for body size by dividing it by the body surface area (m²). This is the cardiac index (normal value 2.5 to 4.0 l/min/m²). Cardiac output and cardiac index reflect the overall efficiency of myocardial performance.

Stroke volume (SV), can be determined from contact bioimpedance measurements by the calculation:

SV=(L³/k)(VET)((dZ/dt)/(Zo))

where VET is ventricular ejection time, L is thoracic length, k is a scaling factor and the bioimpedance measurement gives Zo and dZ/dt.

Cardiac output (CO) is given by the relation:

CO=SV×HR/1000 where HR is heartrate.

The reflections of the radio interrogation signal, like the transmitted voltage test signal used in conventional, contact impedance measurement, have both a constant/baseline component (zero frequency comparable to Zₒ) and a component that varies relatively slowly over time (about 100 Hertz or less), with at least first and second order components (comparable to ΔZ/Δt). For that reason, the same terms, Zₒ, ΔZ/Δt and the like, will be used hereafter in describing the comparable characteristics in the reflected radio interrogation signal. The term “radio frequency impedance interrogation” or “RFII” also will be hereinafter used to refer to constant (zero frequency) and time varying components of the reflections of the radio interrogation signal from the subject. The term “impedance” refers at least to the characteristic of radio interrogation signal changes due to encounters with different bodily substances of different electrical conductivity and changing state, but not the strict electrical meaning of voltage divided by current since the reflected radio interrogation signal is affected by more factors than the conventional contact impedance voltage signal.

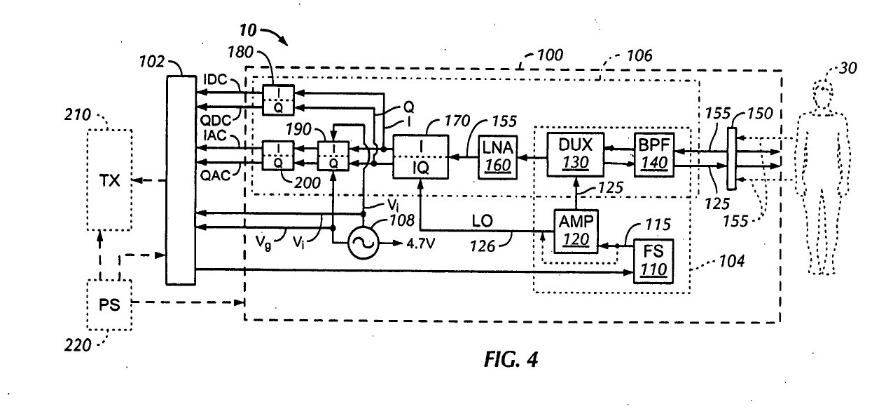

FIG. 4 depicts in functional block diagram form, a presently preferred radio interrogation system. Both technologies, the presently preferred radio interrogation or “RFII” system of the present invention and the conventional conductive impedance systems, can be tested noninvasively on patients simultaneously, without any difficulty or compatibility problems, in order to make a real time comparison of data as reflected by the traces in FIG. 1.

It is helpful in understanding the basic principles of both systems in the comparison measurements to understand similarities and differences in the waveform. In a cardiac impedance measurement using the conventional conductive impedance technology, a low frequency (e.g. 100 kHz), low magnitude (e.g. 4.0 milliamps) alternating voltage signal is introduced into the thorax through a pair of transmitting or “injecting” thoracic electrodes. Two other “sensing” or receiving thoracic electrodes are similarly applied between the injecting electrodes and around the heart to measure the change in voltage associated with the volume and rate of change in the blood flow in the ascending aorta which occurs during the cardiac cycle. The actual voltage change detected by the two receiving electrodes occurs at very low frequencies, from about 1 hertz to less than 100 hertz.

In the present invention, an unmodulated radio interrogation signal of predetermined fixed frequency is transmitted to and into the thorax of a living subject, preferably through a single antenna positioned proximal to the subject, and reflections of the transmitted radio interrogation signal altered from passage through and reflection from various bodily substances within the torso of the subject captured by a radio receiver for processing. FIG. 5 illustrates diagrammatically the various major thoracic reflection surfaces generating the reflections of the radio interrogation signal: derma (D1), muscle (M1), skeletal (S1), lung (L), myocardium (CM), cardiovascular fluid (CF), more skeletal (S2), more muscle (M2) and finally more derma (D2).

The non-invasive, non-contact, radio interrogation apparatus of the present invention, uses a radio signal at a much higher frequency is used than is used to measure cardiac impedance. It has been found that ultra high frequency signals work well, suggestedly between about 900 and 930 MHz, more particularly between 902 and 928 MHz. in the Instrument, Scientific and Medical (“ISM”) band, and more desirably between about 910 and 920 MHz. centered around 915 MHz the center of the ISM band. However, it should be recognized that other radio frequencies would work as well. In addition to being an unregulated band, the ISM band permits the use of reasonably sized patch antennas.

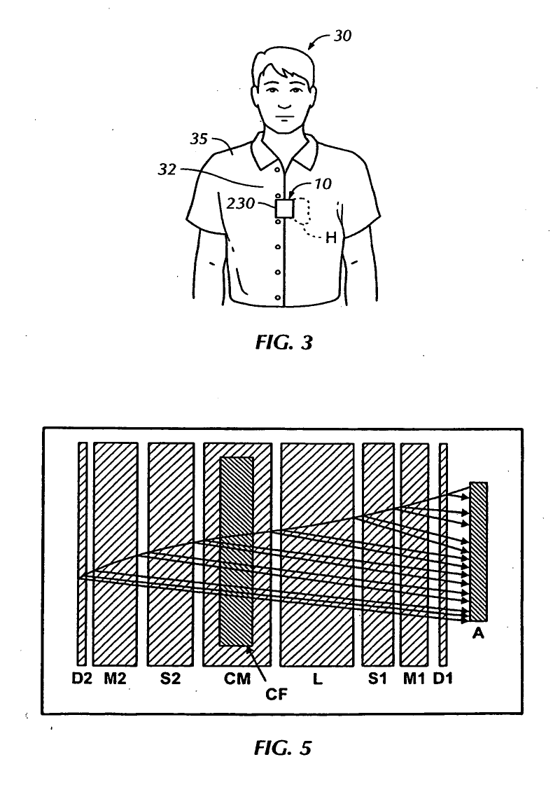

FIG. 4 shows in block diagram form, a presently preferred RFII system indicated generally at 10. The radio apparatus 100 portion of the system 10 for non-invasive radio collection of date of the system 10 include a transmitter portion or “transmitter” indicated generally 104, a receiver portion or “receiver” indicated generally at 106 that partially overlaps the transmitter 104, a reference voltage source 108, and a transmitting/receiving antenna 150. These are used with processing circuitry 102 such as a microprocessor configured by software and/or firmware, to provide the control and preferably impedance data processing portion of the apparatus 100. Another transmitter 210 (in phantom) optionally can be provided to transmit raw or processed data to a remote location. All of the foregoing components are sufficiently low power consumers that all can be packaged together in a palm sized housing 230 (FIG. 3), sufficiently compact to be positioned proximally to the subject 30 and powered by an internal battery power supply (“PS”) 220 (in phantom FIG. 4).

More particularly, the presently preferred radio apparatus 100 includes a precision tone generator as a frequency source (“FS”) 110, a power amplifier (“AMP”) 120 that constitute the basic transmission device, a duplexer (DUX) 130, an RF Band Pass Filter (BPF) 140, antenna 150, a Low Noise Amplifier (LNA) 160, a Demodulator (DUX) 130 that constitutes the basic receiving device, Low Pass filters 180 and 200 and High Pass filters 190 for in phase (I) and quadrature (Q) RFII signals. This baseline hardware can be implemented using commercially available surface mounted RF and mixed signal integrated circuits and components on a multilayer printed circuit board. Apparatus 100 outputs two RFII signals (from demodulator 170) to the processing circuitry 102, an in-phase signal I and a quadrature signal Q. The constant (DC) component of the RFII signals (I DC and Q DC) are comparable to Zo and are output from filter 180 while the time varying component of the RFII signals (I AC and Q AC) are output from the filters 190, 200.

The presently preferred RFII system 10 and radio apparatus 100 uses a single, palm sized, patch antenna 150 (FIG. 3), for example, a fractional wave antenna about 3″×3″, to both transmit and receive the radio signals. Still referring to FIG. 3, the antenna 150 is placed proximal the subject 30, more particularly on the subject's chest proximal the subject's heart H and suggestedly opposite the center of the sternum, where it is aligned juxtaposed with the aorta. The antenna 150 can be placed on the patient's clothing 35 as no direct skin contact is required by the present method and apparatus and clothing of natural or polymer materials does not affect passage of the radio waves. The antenna 150 need only be sufficiently close to the thorax and aorta of the subject to receive usable reflections of the radio interrogation signal transmitted from the patch antenna at a safe power level, for example, about one milliwatt. It has been found that usable reflected signals can be received from the antenna 150 spaced up to about 10 mm from the subject's chest even when the radio interrogation signal is transmitted from antenna 150 at a strength of about one-half milliwatt. However, it is further noted that positioning or movement off the center of the sternum or up or down the sternum can perceivably reduce the signal strength of the received reflections. Therefore, if positioning or movement of the antenna 150 becomes a problem during use, the antenna 150 can be positioned in or under the patent's clothing or even adhered to the patient over the sternum. Again, contact with the subject is not required for the system to work. Furthermore, the apparatus can be operated intermittently, if desired, as changes in hemodynamic data and/or other bodily fluid data are much slower than the cycling frequency at which the apparatus 100 is capable of operating. For example, only twenty-five percent duty cycles of appropriate thirty second lengths need be run to long term monitor a subject's condition.

When operating, the presently preferred radio apparatus 100 of the RFII system 10 described herein runs on “full duplex”, meaning that the radio interrogation signal is being transmitted and the reflections of that signal are being captured simultaneously through the same antenna 150. Overlap is accomplished through duplexer 130 in FIG. 4 that separates the transmitted and received radio signals. The need for only a single signal transmitter and single signal receiver with only a single antenna are important characteristics of the present invention that distinguish it from conventional contact impedance systems.

Again, it has been discovered that the captured reflections of the radio interrogation signal have a constant (zero frequency) components and a component that changes relatively slowly with time relative to the transmitted radio interrogation signal's amplitude and predetermined fixed frequency that relate to and mimic the impedance changes detected by conventional contact impedance measuring systems. The processing circuitry 102 in FIG. 4) preferably is configured by firmware or software to the cycling of the radio apparatus 100 and is further configured to at least temporarily store the RFII signals and preferably process the RFII signals to determine at least one cardio-respiratory characteristic of the subject to measure and/or monitor. These characteristics that can be measured and monitored from this radio interrogation apparatus include but are not limited to heart rate, respiratory rate, stroke volume and cardiac output. Since radio measured “impedance” is also dependant on the overall conductivity and absorptiveness of the body's blood and tissue, it is dependent on vital chemical conditions that can change RF conductivity, such as body hydration, or deficient oxygen content. In particular, the more electrically conductive substances such as blood are more reflective of radio waves than are the less conductive tissues. The RFII signal has a constant or DC component of the signal is the equivalent baseline impedance, Z_{O}. The moving parts of the heart and the blood flow also cause the amplitude and phase of the reflected radio signal to change over time at a very low frequency determined, in part, by the cardiac cycle. This very low frequency pattern includes reflects ΔZ/Δt. That is, the mechanical motion of the heart and blood flow, relative to the antenna frequency, modulates the RFII signal with a frequency modulation (FM) content of about 1 to 100 Hz, in addition to amplitude modulation. The receiver portion 106 of the present apparatus 100 extracts both equivalent Z_{O} and ΔZ/Δt impedance components from the captured RFII signal reflections and preferably forwards them to the processing circuitry 102 for quantification and analysis.

The reference voltage source 108 preferably is implemented by a low noise drop-out linear voltage regulator (not separately depicted), which supplies 4.7V to all of the circuitry except the High Pass filters 190. These receive isolated power supplies Vᵢ, V_{q}, suggestedly 2.5V each. Two 4.7V sources can be provided, if desired, one continuous and one switch controlled, so that some of the components can be powered down when not in use. Suggestedly, only the High Pass filters 190 need be continuously powered.

The frequency source (FS) 110 producing the RFII test signal generates a precision unmodulated voltage signal, a pure UHF tone, that does not introduce any relatively low frequency (e.g. less than about 100 Hz) noise content, which cannot be extracted by the receiver portion 106 of the apparatus. Lowering electronic noise from the frequency source, especially ultra and extremely low frequency “1/f” or “phase noise” (at least from 1.0 to 100 Hz), as much as can practically be done, is a key part of lowering RFII electronic noise and improving RFII receiver sensitivity. The frequency source signal is split and amplified to produce two ultra-high frequency reference signals. One becomes the radio interrogation signal that is transmitted into the subject via the duplexer 130 and antenna 150. The other signal goes to the demodulator/receiver 170, where it becomes the local oscillator (LO) used for the RF frequency down conversion.

In the presently preferred design, the frequency source 110 includes a frequency synthesizer to generate the pure tone signal (represented by reference number 115) of predetermined fixed frequency, for example, 915 MHz. The described RFII system 100 uses 915 MHz because it lies at the center of the 902 MHz to 928 MHz Instrument, Scientific, and Medical (ISM) frequency band, a special frequency band set aside where specific governmental licensing (e.g. FCC) is not required. The invention is not limited to the 915 MHz frequency or even the 902 MHz to 928 MHz ISM frequency band. This frequency synthesizer is preferably provided with a four order PLL loop filter designed for the noise performance and frequency switching time requirements of the apparatus. Since the 915 MHz signal 115 is not to be switched in frequency, the loop filter can be optimized for low electronic noise only. An outside “tank” or precision passive resonant circuit is preferably provided following the four order PLL loop filter.

Power amplifier 120 is operably connected between source 110 and antenna 150. It has been found that only a relatively small amplification of the frequency source signal 115 is needed to boost signal strength to the antenna 150 sufficient to make an accurate measurement at a safe power level. Suggestedly, power amplifier 120 boosts the frequency source signal sufficiently to generate a radio interrogation signal (represented by reference number 125) transmitted from the antenna 150 at a strength no greater than about one milliwatt, a level deemed safe by regulation. In the described apparatus, the power amplifier is configured to preferably amplify the frequency source signal sufficiently to transmit the RFII signal 125 from the antenna 150 at a strength of only about 0.5 milliwatts or −3 dBm on a decibel RF power scale. In the described system, the Band Pass Filter (BPF) 140, duplexer (DUX) 130 and RF transmission lines and connectors (unnumbered) will cause total loss of about 7.0 decibels (dB) between power amplifier 120 and the antenna 150. The power amplifier 120 should overcome the cumulative signal loss caused by the duplexer (DUX) 130 and band pass filter (BPF) 140 insertion loss, so the RFII signal is at the design transmit level (e.g. 0.5 watt/−3 dBm) at the antenna 150. Power amplifier 120 is preferably a differential output VCO buffer amplifier that amplifies the frequency source signal 115 and passes that signal to the remainder of the transmitter portion 104 of the system 100, and provides a reference local oscillator (LO) signal (represented by reference number 126) for the receiver portion 106. More particularly, a power amplifier 120 is used which provides two signals, a first signal used as the radio interrogation signal 125 and an inverse signal of reversed phase (180 degree phase shifted) used as the receiver local oscillator LO signal 126. Both signals are exactly the same frequency (915 MHz) at about +4 dBm output RF power each.

The duplexer 130 is operably connected between power amplifier 120 of the transmitter 104 and Low Noise Amplifier 160 and demodulator 170 of the receiver 106. The duplexer 130 is suggestedly a 4-port 3 dB quadrature (90° phase offset) hybrid configured from a S03B888N3 chip. Duplexer 130 sends an RFII signal 125 (Q) received at a port 1 from power amp 120 out to the antenna 150, which is connected to port 2, while preventing transmitter power in port 1 from going to the remainder of the receiver portion 106 of the system 100 connected to port 3. The unused 4th port is loaded with a fifty ohm termination. Duplexer 130 allows full duplex radio operation, which is the ability to use the same antenna 150 for simultaneously transmitting and receiving. Ideally a duplexer should have very high isolation to prevent RF transmitter signal from leaking into the receiver, becoming a potential source of noise and received signal error, and low insertion loss for the signal passing through it. There is a total approximately 6 dB loss from transmitted radio interrogation power to the received reflections of that signal at the receiver 106 (round trip path) using this duplexer 130. The identified duplexer 130 suggested has a minimum 20 dB isolation and a maximum insertion loss of 3.15 dB.

Finally, RF bandpass filter (BPF) 140 is operably connected between the power amplifier 120 of transmitter 104 and the antenna 150. BPF 140 is designed to allow only the transmitted signal frequency, 915 MHz for example, to pass and to exclude any harmonic frequencies generated by amplification to be transmitted. Suggestedly, a passive dielectric filter, designed for the 902 MHz to 928 MHz ISM band is used. Filters operating in this band are available having an insertion loss of about 2.2 dB and about a 3% bandwidth, with respect to the 915 MHz carrier signal. The bandwidth permits it to also pass the transmitted radio interrogation signal with Doppler effect shifts containing cardio-respiratory information reflected back to the antenna 150.

Referring to FIG. 4, the signal from the transmitter portion 104 of the described apparatus 100 is transmitted through the antenna 150 to and into the body of a proximal subject 30, preferably with a −3 dBm/0.5 milliwatt power level. The RFII signal penetrates the subject's body in particular, the subject's thorax 32 (FIG. 3), and is partially absorbed by and reflected off the more electrically conductive body components. The major tissue surfaces that reflect the radio interrogation signal are depicted diagrammatically in FIG. 5. The duplexer 130 separates the transmitted RFII and received reflected RFII signal at the same time. The reflections of the radio interrogation signal will have lower amplitude and power, typically in the range of −20 dBm to −30 dBm, and a different frequency component, which represents the RF “impedance” of the reflected radio signal. The suggested antenna 150 is preferably a palm sized, fractional wave patch antenna that that is sufficiently small to be positionable right up to the chest 32 of the subject 30 (FIG. 3). The close proximity gives the antenna 150 characteristics of a near field RF coupling device or transducer.

The receiver portion 106 of the circuit 100 must capture the reflections of the radio interrogation signals returned from the subject 30 (represented by reference numeral 155 in FIG. 4) and extract the very low, DC to 100 Hz content, representing the impedance equivalent of the radio interrogation signal. In addition, the receiver portion 106 of the circuit 100 should reject unwanted noise in the reflected signals 155 that may have been generated by the transmitter portion 104, as well as minimize internal noise generated by the circuitry of the receiver 106 itself, that would alter the waveform if the reflected signal 155. The receiver portion 106 should have a large signal-to-noise (S/N) ratio indicating a maximal received signal strength, with any interfering noise ideally reduced to zero strength. The receiver portion 106 includes a “front end” with the Band Pass Filter (BPF) 140, the duplexer (DUX) 130, and a Low Noise Amplifier (LNA) 160 and a “back end” with the demodulator 170 and filters 180, 190, 200. Since the suggested BPF 140 is a passive filter, it can be connected directly to the antenna 150 where it can be used for both transmit and receive signal filtering functions. The reflected signals 155 are separated from the transmitted signal through the duplexer 130, and are directed to the low noise amplifier (LNA) 160.

The disclosed receiver design preferably has about a 7.0 dB front end loss: about 2.2 dB at the BPF 140 and about 3.15 dB at duplexer 130, with the approximate remainder lost in the RF transmission line and connector to the antenna 150. The low noise amplifier (LNA) 160 plus the following active LP and HP filters must make up approximately 27 dB of signal gain. The low noise amplifier (LNA) 160 in particular, suggested is fabricated with a semiconductor process that has inherent low phase noise. Silicon Bipolar processes or Silicon Germanium processes are best for low phase noise, slightly better than GaAs or InP, but at least GaAs can be used.

The demodulator 170 is operably connected with the antenna 150 through duplexer 130 and low noise amplifier 160. A quadrature demodulator whose architecture is known as direct conversion quadrature demodulation is preferred. In direct conversion, one down-conversion stage is used. The captured reflected signals 155 from the low noise amplifier 160 are split with no phase shift, and fed to two identical mixers, an in phase mixer (“I”) and quadrature mixer (“Q”), in the demodulator 170. Each mixer also receives the local oscillator (LO) signal 126, where the “I” mixer receives a buffered signal identical to the transmitted signal 125, and the “Q” mixer receives the same buffered signal identical to the transmitted signal 125, but shifted 90° in phase by circuitry provided in the demodulator. The primary mixer output of two input signals 125/126 and 155 is both the sum and difference of the two input signals:

MIXER OUTPUT FREQUENCIES f_{OUT}=(f_{RF}+f_{LO})+(f_{RF}−f_{LO})

Since the same frequency source 104 is used for the original radio interrogation signal 125 and the local oscillator signal 126 of the demodulator, the only difference in the frequencies of transmitted radio interrogation signal f_{TX} (125) and received reflected RFII containing signals f_{RX} (155), is the very low frequency, Doppler modulation Δf_{FM} from DC to about 100 Hz representing the radio interrogation equivalent cardiac impedance where

f_{LO}=f_{TX} and f_{RX}=f_{TX}+Δf_{FM}+δDC

the demodulator/receiver mixer output: f_{MIX}=(Δf_{FM}+δ_{DC})+(2f_{TX}+Δf_{FM})

the demodulator/receiver filter output: f_{OUT}=(Δf_{FM}+δ_{DC})

The mixer output for an ideal direct conversion quadrature demodulator system would be a combination of the low frequency Doppler modulation Δf_{FM}, DC [0 Hz] to 100 Hz, plus a constant DC offset, δ_{DC}, and a redundant high frequency band centered around twice the carrier frequency (i.e. 2f_{TX}=1830 MHz). The filtering easily removes this redundant high frequency component (2f_{TX}+Δf_{FM}). The radio interrogation signal 125 and local oscillator signal 126, though generated identically, have a phase difference of 180° from the differential Q and QB outputs of the transmitter 104/amplifier 120. In addition there is an arbitrary but fixed signal path difference between the local oscillator signal 126 and received reflected signals 155 when mixed together. At the inputs of demodulator 170, the received signal and the local oscillator signal have a resulting arbitrary constant phase difference, θ_{DC}. From this fixed phase difference θ_{DC}, the demodulator 120 will produce an offset DC signal δ_{DC}. The demodulator mixer output must be filtered and buffered at low frequencies (DC −200 Hz) to send the RFII data signal (Δf_{FM}, +δ_{DC}) to the processor 102 preferably for processing to extract the cardio-respiratory information.

For the RFII receiver 106, the demodulator 170 should suggestedly be fabricated in a low phase noise process like Silicon Bipolar or Silicon Germanium (SiGe), the 3rd order distortion (IP3) power level should be relatively high, the LO input should be driven to a relatively high level, and the internal mixers should have good conversion efficiency. As an expedient, a cordless telephone transceiver chip, for example an RF9904 chip, could be used as the demodulator 170. The transmitter portion of the transceiver would not be used. The transceiver can be fabricated in a silicon bipolar process that has low 1/f and phase noise compared to other semiconductor processes. The identified transreceiver has a receiver noise figure of 10 dB, voltage gain of 3 dB, and I and Q output DC level of 3.5 V with a maximum voltage swing of 3.0 V to 4.0 V (1.0 Vpp). The local oscillator maximum power input is +5.0 dBm and it is suggestedly driven at +4.0 dBm in the present design. The maximum input RF signal power to the receiver, without output signal distortion, is less than −2.0 dBm.

Finally, low pass (LP) filtering and buffering is provided for 1 and Q DC and I and Q AC outputs. The first pair of outputs, the I and Q DC pair, is simply the low pass filtered I and Q output of the quadrature demodulation, represented by the signal

f_{OUT}=(Δf_{FM}, +δ_{DC})+(2f_{TX}+Δf_{FM})

The LP filters 180 (I and Q) are preferably passive, RC filters that remove any high RF frequency residue component (2f_{TX}+Δf_{FM}) from the I and Q outputs of demodulator 170. The resulting I and Q DC pair carry the data signal (Δf_{FM}, +δ_{DC}) with a frequency content of DC (0 Hz) to 200 Hz. The LP filter(s) 180 used may actually be configured with a relatively high cutoff (e.g. 5 MHz) as there is no appreciable source of electronic noise between 200 Hz and 5 MHz in the above-described design. The main purpose of the I and Q DC outputs are to retain the DC signal which contains the base equivalent thoracic impedance component (Zo). The DC signal component of the I and Q DC outputs, δ_{DC}, contain a DC voltage representing the baseline impedance value, δ_{ZDC}, and an error DC voltage δ_{Δθ} representing the fixed phase difference between the receiver input (radio interrogation reflections) and local oscillator signals 155, 126 discussed above.

δ_{DC}=δ_{ZDC}+δ_{Δθ}

The I and Q DC signals must be processed digitally to remove the frequency content and to remove the added receiver DC phase difference error δ_{Δθ} to produce the equivalent base impedance component Zo. This may be done by measuring the fixed input phase difference between the RF and LO inputs of the demodulator 170 that are part of the circuitry through a calibration procedure done at manufacture, with a DC offset calibration factor provided in the processing circuitry 102 for any digital signal processing. The I and Q AC outputs are the I and Q mixer outputs that undergo band pass filtering, resulting in a signal frequency content between of 1 Hz and 100 Hz. These signals will contain the time varying portions of the equivalent cardiac impedance signal given as,

ΔZ/Δt=dZ/dt+d²Z/dt²

The I and Q mixer output signals from demodulator 170 are preferably first put through a pair of identical, active, high pass (HP) filters 190 (I and Q) that are designed to block frequencies below about 1, preferably below 1.0 Hz. This is followed by a pair of identical low pass active (LP) filters 200 (I and Q) that are designed to block all frequencies above 200 Hz. or, more particularly, pass all remaining frequencies below about 200 Hz. Each of the presently preferred HP active filters 190 (I and Q) includes an RC filter with a low noise, buffer amplifier preferably supplied with an isolated 2.5 Volt reference voltage (Vi and Vq) and each has a designed signal gain of G=10. Op amps of the voltage source 108 buffer and isolate the 2.5 V references Vi and Vq from one another and the 4.7V system voltage.

The presently preferred LP filters 200 (I and Q) are active configurations known as Multiple Feedback Topography (MFT). This active filter type is selected for its high Q factor and gain for low distortion and ability to provide gain for an output signal driver. Each LP filter 200 (I and Q) includes an op amp with RC components. This MFT is modified to be referenced to the Vi/Vq 2.5V reference voltages. Both reference voltages Vi and Vq can be output to the processing circuitry 102 as reference DC voltages. Preferably, at least six output signals are provided from the radio portion 100 of the system 10 to the processing circuitry 102 for calculation of equivalent thoracic impedance and other cardiopulmonary data: I DC and Q DC, I AC and Q AC, and reference DC voltages Vi and Vq. These signals are digitized and used by processing circuitry 102 to determine the equivalent Z_{O}, ΔZ/Δt and d²Z/dt² and other values from those equivalents by various processing methods.

A benefit of separate I and Q outputs is that with signal processing, the received signal can be enhanced using both amplitude and phase information, and still effectively remove signal noise. A first simple signal processing algorithm is presented here, directed to enhancement of the cardiac waveform signal.

The signal coming out from the demodulator 170 and filters 180, 190, 200 can be thought of as a 3-D signal with an I component, a Q component, and time. It is difficult for the user to see and interpret a 3-D display or two 2D displays with both I and Q signals superimposed on top of each other. The method selected is very analogous to an FM polar discriminator, which uses I and Q demodulated signals.

If quadrature-phase signal Q were plotted with respect to the in-phase signal I, this would result in a roughly long and thin ellipse pattern. A line through the center of the I and Q ellipse will form an angle Φ with respect to the horizontal (I) axis. The I and Q data points can be plotted in polar form as follows,

RIᵢ=SQRT(I²+Q²)*[cos (Φ)]RQᵢ=SQRT(I²+Q²)*[sin (Φ)]

Where Φ=tan⁻¹ (Qᵢ/Iᵢ), and function tan⁻¹ is defined from −π to +π radians (−180° to +180°). (If function tan⁻¹ were defined from only −π/2 to +π/2 radians (−90° to +90°) in a computer language, the sign of the I and Q components would have to be identified to determine the angle 180° to +180° over all angles with no ambiguities, a seemingly trivial but important detail.) Also a denominator value I₁ that is close to zero, less than the least significant bit, is desired to avoid a divide by zero event in the algorithm.

If the I and Q signal are rotated without changing amplitude by a correcting angle θ=−Φ then the signal would have a maximized amplitude along the horizontal I axis, and the rotation phase angle θ is known when I is maximized and Q is minimized. The rotated I and Q signal is plotted as

RIᵢ=SQRT(I²+Q²)*[cos (Φ)+θ)]RQᵢ=SQRT(I²+Q²)*[sin (Φ+θ)] and, when rotated so θ=−Φ, then the signal becomes

RIᵢ=SQRT(I²+Q²)RQᵢ=0.

At this point the constant phase θ represents a constant DC value, the baseline cardiac impedance Z_{O} equivalent, plus the arbitrary phase offset between the reflected RF signal from the low noise amplifier 160 and the local oscillator signal of the demodulator 170. If a calibration measurement is made where the antenna was replaced with a 10 dB RF load and short circuit, the measured rotational angle φₑᵣᵣ would represent only the arbitrary phase error. Then when a patient's thorax is measured, the phase θ_{Z} representing Z_{O} can be extracted.

θ=θ_{Z}+φₑᵣᵣ, θ_{Z}=θ−φₑᵣᵣ

The time varying component or the derivative of phase offset (dθ_{Z}/dt ), which is the Doppler frequency, is found by an iterative method where the signal phase is sampled at least twice as fast as the highest possible FM frequency (i.e. 200 times per second for 100 Hz data). This iterative process yields the Doppler frequencies, representing the equivalent time varying cardiac impedance wave form ΔZ/Δt. This is accomplished as follows.

Find initial I and Q rotation:

RIᵢ=SQRT(I²+Q²)*[cos(Φ)]RQᵢ=SQRT(I²+Q²)*[sin (Φ)],

Rotate by angle θ where signal I is maximized and signal Q is minimized.

RIᵢ=SQRT(I²+Q²)[cos(Φ+θ)]=RIᵢ=SQRT(I²+Q²)

RQᵢ=SQRT(I²+Q² [sin(Φ)]=0

Then we can find, θ_{Z}=θ−φₑᵣᵣ.

Now there is a new measurement “I” for Iᵢ and Qᵢ, and the previous measurement is set above to “i−1”, where we write Iᵢ₋₁ and Qᵢ₋₁. Upon update of the rotation angle, we find the iterative angle Δθ is:

Δθ=θᵢ−θᵢ₋₁=tan⁻¹{(Qᵢ−Qᵢ₋₁)/(Iᵢ−Iᵢ₋₁)}

When large angles are measured, there may be scaling problems since ratio Iᵢ/Qᵢ is very large when angle Φ=tan⁻¹ (Qᵢ/Iᵢ) approaches 90° and Q approaches zero. The calculation can be weighed for the iterative angle by the square of the distance between the I and Q measurements,

Errᵢ[(Qᵢ−Qᵢ₋₁)²+(Iᵢ−Iᵢ₋₁)²]*tan⁻¹ {(Qᵢ−Qᵢ₋₁)/(Iᵢ−Iᵢ₋₁)}

Using a scaling factor “A”, (typical value A=0.0005) we measure a large angle is measured as:

θᵢ=A*Errᵢ

The iterative angle Δθ, is updated, by subtracting the previous angle

Δθ=θᵢ−A*Errᵢ₋₁ or in programming form, θᵢ=θᵢ−A*Errᵢ₋₁

The value Δθ will vary over time, representing the Doppler shift frequencies and the cardiac impedance. Amplitude of the Doppler signal is recovered for both phase and amplitude information. This algorithm was chosen since it can provide for a higher signal to noise ratio in a receiver, and by scaling, it can deal with large dynamic range. Over time, other algorithms may be developed that will further enhance the cardiac impedance waveform, and extract more information.

It has been found that there is clear separation between the RF analog transmitting and receiving sections and low frequency IF section and signal processing. In an ASIC design it may prove better to have two ASICs—with one optimized for the RF receiver functions and a second digital chip, optimized for digital signal processing, memory and device management functions. The second may even replace some of the analog active filtering described above, doing all analog and signal processing in a flexible digital processing environment.

PCT/US2007/020492 filed Sep. 21, 2007, entitled “Antenna for Thoracic Radio Interrogation”, U.S. Provisional application No. 60/846,408 entitled “Transducer-antenna-probe for Thoracic Radio Interrogation”, filed Sep. 21, 2006; U.S. Provisional Application No. 60/910,394, entitled “Antenna for Thoracic Radio Interrogation”, filed Apr. 5, 2007; and U.S. Provisional Application No. 60/973,970, entitled “Antenna for Thoracic Radio Interrogation”, filed Sep. 20, 2007; PCT/US2007/020487 filed Sep. 21, 2007, entitled “Method for Processing Thoracic Reflected Radio Interrogation Signals”; and PCT/US2007/020486 filed Sep. 21, 2007, entitled “Relative Positioning System And Method” are all also incorporated by reference herein in their entireties.

It will be appreciated by those skilled in the art changes could be made to the embodiments described above without departing from the broad inventive concept thereof.

We claim:

1. A radio apparatus for non-invasive, in-vivo, thoracic radio interrogation of a subject to collect hemodynamic, respiratory and/or other cardiopulmonary related data from the subject comprising:
an antenna sized to be positioned proximally to the subject outside the subject;
a radio transmitter operably connected to the antenna and configured to transmit only an unmodulated radio interrogation signal of a predetermined, fixed frequency through the antenna and into the proximally positioned subject; and
a radio receiver operably connected to the antenna and configured to capture through the antenna reflections of the transmitted radio interrogation signal returned from the subject.

2. The radio apparatus according to claim 1 wherein predetermined fixed frequency of the radio frequency impedance interrogation signal is an ultra high frequency.

3. The radio apparatus according to claim 1, wherein the ultra high frequency is between 900 and 930 MHz.

4. The radio apparatus according to claim 1, wherein the radio frequency interrogation signal broadcast from the antenna essentially lacks noise components below at least about one hundred Hertz.

5. The radio apparatus according to claim 1 wherein the radio transmitter comprises a source of a signal of the fixed predetermined frequency and wherein the radio transmitter comprises an amplifier operably connected between the source and the antenna to amplify the source signal sufficiently to transmit the radio interrogation signal from the antenna at a strength no greater than about one milliwatt.

6. The radio apparatus according to claim 1 further comprising a duplexer operably connected between the radio transmitter and the antenna and between the antenna and the radio receiver for simultaneous operation of the radio transmitter and the radio receiver through the antenna.

7. The radio apparatus according to claim 6 wherein:
the radio receiver includes a quadrature demodulator operably connected with the duplexer; and
the quadrature demodulator outputs at least one signal containing Doppler components extracted from the reflections of the radio interrogation signal.

8. The radio apparatus according to claim 7 wherein the radio receiver further comprises a high pass filter operably connected to the quadrature demodulator and configured to pass Doppler components extracted from the reflections of the radio interrogation signals and having frequencies above about one hertz.

9. The radio apparatus according to claim 18, wherein the radio receiver further comprises a low pass filter operably connected to the high pass filter and configured to pass Doppler components extracted from the reflections of the radio interrogation signals and having frequencies up to only about one hundred hertz.

10. The radio apparatus of claim 7 further comprising processing circuitry operably coupled with the quadrature demodulator so as to receive at least the Doppler components extracted from reflections of the radio interrogation signal.

11. The radio apparatus of claim 1 further comprising a palm size housing containing the radio transmitter, the patch antenna and the radio receiver, the transceiver being sufficiently light to be hand carried and placed proximal the subject.

12. The radio apparatus of claim 1 wherein the antenna is a patch antenna.

13. A method for non-invasive, in-vivo, thoracic radio interrogation of a subject to collect hemodynamic, respiratory and/or other cardiopulmonary related data from the subject comprising the steps of:
positioning an antenna proximally to the subject transmitting only an unmodulated radio interrogation signal of a predetermined fixed frequency through the antenna and into the proximately positioned subject; and
capturing through the antenna with an operably connected radio receiver reflections of the transmitted radio interrogation signal returned from the subject.

14. The method according to claim 13 where the transmitting step comprises transmitting the radio interrogation signal at an ultra high frequency.

15. The method according to claim 13 where the transmitting step comprises transmitting the radio interrogation signal at a frequency between 900 and 930 MHz

16. The method according to claim 13, wherein the transmitting step comprises transmitting the radio interrogation signal essentially without noise components below at least about one hundred Hertz.

17. The method according to claim 13 wherein the transmitting step comprises transmitting the radio interrogation signal from the antenna at a strength no greater than about one milliwatt.

18. The method according to claim 13 further comprising the step of duplexing the transmitting and capturing steps though the patch antenna.

19. The method according to claim 18 further comprising the step of filtering a duplexed radio interrogation signal before broadcasting the radio interrogation signal from the antenna.

20. The method according to claim 18 wherein the duplexing step comprises passing to and from the patch antenna only ultra high frequency signals centered around the predetermined fixed frequency.

21. The method of claim 13 wherein the capturing step comprises extracting Doppler components from the captured reflections of the radio interrogation signals.

22. The method according to claim 13 wherein the extracting step comprises extracting components from the reflected radio interrogation signals having frequencies between about one and 100 hertz.

23. The method according to claim 13, further comprising the initial step of combining the antenna with a radio transmitter to perform the transmitting step and a radio receiver to perform the capturing step all in a palm size housing.

24. The method of claim 23 wherein the positioning step comprises placing the palm size housing on the subject to perform the transmitting and capturing step.

25. The method according to claim 23 wherein the positioning step is performed by placing the housing on clothing of the subject separating the housing from direct contact with the subject.
